(19) United States
(12) Patent Application Publication
(10) Pub. No.: US 2002/0119580 A1
(43) Pub. Date: Aug. 29, 2002

(54) Radiofrequency encoded chemical library synthesis particles method

(76) Inventors: Anthony Robert Corless, Middlesex (GB); David Andrew Wenn, Middlesex (GB)

Correspondence Address: PILLSBURY WINTHROP, LLP, P.O. BOX 10500, MCLEAN, VA, 22102, US

(21) Appl. No.: 09/971,112

(22) Filed: Oct. 5, 2001

Related U.S. Application Data
(63) Continuation of application No. 09/403,202, filed on Oct. 15, 1999, now abandoned.
Application No. 09/403,202 is a 371 of international application No. PCT/GB98/01064, filed on Apr. 14, 1998.

(30) Foreign Application Priority Data
Apr. 17, 1997 (GB) 9707744.0

(51) Int. Cl. G01N 33/53; G01N 33/566; G01N 33/543; H04B 17/00

(52) U.S. Cl. 436/518; 435/7.1; 436/501; 455/226.1

(57) ABSTRACT

A radiofrequency encoded chemical library synthesis particle which comprises a read-only radiofrequency tag linked to a solid phase. Chemical libraries synthesised on such particles and their use in biological screening methods.

### METHOD

Chemical libraries may be assembled by a number of methods, including the ‘combine/mix/divide’, or split synthesis process described by Furka et al (Abstr. 14th int. Congr, Biochem., Prague, Czechoslovakia, 1988, 5, 47; Int. J. Pept. Prot. Res, 1991, 37, 487-493) for creating libraries on polymer beads, in which each bead contains one discrete chemical species. The individual components of the library may. be tested either still attached to the polymer bead on which they were synthesised (Lam et al, Nature, 1991, 354, 82-84) or after cleavage from the bead (Salmon et al, Proc. Nat. Acad. Sci. USA, 1993, 90,11708-11712). If tested while attached to the bead, or cleaved but physically associated with the bead, it is necessary to devise a method of identifying the chemical which is bound to any bead found to be biologically active in the test. Where this compound is a polypeptide this may be achieved by Edman degradation, either directly or after cleavage from the bead (Lam et al, Bioorg. Med. Chem. Lett., 1993, 3, 419-424); oligo nucleotides may be identified by microsequencing techniques (Dower et al, Ann. Rep. Med. Chem., 1991, 26, 271-280). Other small molecules may be identified directly by electrospray, matrix-assisted laser desorption, or time-of-flight secondary ion mass spectrometry techniques, (Brummel et al, Analyt. Chem., 1996, 68, 237-42).

Researchers have attempted to identify peptides containing unnatural amino acids, which are not amenable to Edman degradtion by co-synthesising a second peptide chain comprising natural amino acids and using this as as a sequenceable ‘code’ (Nikolaiev et al, Peptide Research, 1993, 6, 161-170), and others have used oligonucleotide chains as ‘codes’ to identify the other ligands (Needels et al, Proc. Nat Acad. Sci. USA, 1993, 90, 10700-10704), while mixtures of halogenated aromatic compounds have been used, incorporated in trace amounts at each stage of the synthesis; to formsn identifiable (by gas chromatography) ‘binary code’ system for ligand definition (Borchardt and Still, J. Am. Chem. Soc., 1994, 116, 373-374). These methods have been reviewed extensively (Jacobs and Fodor, TIBTECH, 1994, 12, 19-26; Pavia et al (Eds), Bioorg. Med. Chem. Lett., 1993, 3, 381-470; Moos et al, Ann. Rep. Med. Chem., 1993, 28, 315-324; Gordon et al, J. Med. Chem., 1994, 37, 1233-1251, and 1386-1401; K. D. Janda, Proc. Natl. Acad. Sci. USA, 1994, 91, 10779-10785).

An alternative to such chemical coding or tagging strategies has been the use of silicon chips in the form of radiofrequency, or ‘RF’ tags, which, when associated with self-contained packets of a number of polymer beads, can be used to store information about the chemical synthetic processes used to make a particular ligand, and hence by inference, the chemical structure of the resultant ligand (Moran et al, J Am. Chem. Soc., 1995, 117, 10787-10788; Nicolaou et al, Angew. Chem. Int. Ed. Engl., 1995, 34, 2289-2291). An advantage of this RF approach is that by using an essentially non-chemical tag, the risk of the vital stored information being affected, or at worst destroyed, by the chemical synthetic processes used to construct the ligand is considerably lessened. However, a principal disadvantage to the methods already described (vide supra) is the size of the RF tag device that is used.

For example, Nicolaou et al (op cit) used a semiconductor RF device measuring alone 8×1×1 mm, excluding the inductive coil. Hence, the size of package containing the RF tag and the synthesis resin beads is large and actual physical handling of the packages is somewhat unwieldy. In addition, the cost of each individual package is appreciable

We have now devised new radio-frequency encoded particles for use in chemical library synthesis and deconvolution.

In a first aspect of the invention we provided a method for the preparation of a chemical library which method comprises synthesising the library on a plurality of radiofrequency encoded particles, each particle comprising a read-only radiofequency tag, so as to provide a chemical library comprising a plurality of tagged particles to each of which is attached at least one member of the library.

Library synthesis is conveniently effected by the so-called split-synthesis, split-and-mix or one-compound-one-bead process originally described by Furka et al (Abstr. 14th Int. Congr. Biochem., Prague, Czechoslovakia, 1988, 5, 47) and further exemplified by Lam et al (Nature, 1991, 354, 82-84). In summary, this process involves dividing or ‘splitting’ a pool of solid-support particles, for example resin beads, into separate vessels, then reacting the particles in each vessel each with a different reagent or building block, allowing the reactions to proceed to completion, then ‘mixing’ the particles from each vessel to generate a second pool of particles which is split, reacted and mixed in the same way as above. The consequence of this process, which does not involve the use of mixtures of building block reagents, is that only one compound appears on any one particular synthesis particle.

Conveniently the library comprises no more than five compounds per bead, more conveniently one compound per bead.

By the term “radiofrequency encoded particles”, or ‘RF beads’, we mean discrete, insoluble beads or solid support particles that participate in the library synthesis and which individually, carry a discrete ‘tag’ in the form of an electromagnetically powered silicon integrated circuit, or ‘chip’ which is itself carried on an inert substrate that is inextricably associated with a chemical synthesis polymer or resin, which is an integral part of the RF bead. The RF encoded particles or beads may or may not be spherical. In this patent application the terms “particle” and “bead” are used interchangeably.

By the term “read-only” we mean a tag used in such a way that during the use of the tag to monitor and track chemical processing the data held on the tag is unchanged. The use of a writeable tag to which data is written as a precursor to the process, but not modified as a means of recording the process steps, is considered as a “read-only RF tag” since that is the way in which the tag is used during the processes of interest.

By the term read-only RF tag we mean an integrated circuit that has had incorporated into its memory during manufacture a serial number or code which can be read during the process of compound library production, but whose memory cannot be fiber written to or programmed easily after particle production is complete. In other words the tag possesses read-only memory, or ‘ROM’. This ROM is preferably a mask to program a set of conducting links within the structure of the device, since such structure is unlikely to deteriorate in the chemical process conditions envisaged. We also disclose the use of a writeable device such as EEPROM, to which a unique number is written prior to the start of the chemical processing.

The radiofrequency encoded particles are tracked during library synthesis. That is to say particles can be identified at any given time and their progress through a particular chemical synthesis regime is recorded The overall purpose of tracking is to associate the identity of a particular bead with the chemical synthesis pathway. By way of non-limiting example, a RF encoded particle is picked up, rotated or otherwise manipulated so as to be conveniently presented to a suitable device for reading and recording of the particular RF code stored in the chip's memory; then deposited in a locus of choice. A locus may be a reaction vessel, a mixing vessel, an assay vessel or any other container that is used in the compound library synthesis and testing procedure. The directing and recording process establishes an association or connection between any one particular bead, as defined by its RF code or tag, and a particular locus.

By the movement of each particle in the synthesis procedure we mean the progress of each RF encoded particle from one locus to the next. The essential feature is that when the library is in the divided state, the association of each particle with its locus must be determined. Typically, each particle will be recorded as it enters or leaves each locus. However the record of movement may be made at any time whilst a particle or subset of particles is either destined for a particular locus, or has been the subject of a particular reaction or process within that locus. As each RF encoded particle passes through different loci, the association between a particular movement history or ‘audit trail’, and a particular RF code is established.

In a further aspect of the invention we provide a. chemical library which comprises a plurality of radiofrequency encoded particles, each particle comprising a read-only radiofrequency tag and having at least one member of the library attached thereto.

The chemical library may be used in screening methods to identify compounds which modulate the activity of a biological of interest. Typically, a compound will be cleaved from its associated RF encoded particle before testing; alternatively, compounds are tested whilst still attached to their particles. In both cases, there needs to be an association between the measured activity and the particle that gave rise to that activity. Once a particle of interest is identified, its RF code is read, preferably in substantially the same way as in the course of the synthesis. This RF tag is then compared to the record of the movement of the particles made during the synthesis procedure, and the structure of the compound of interest is inferred.

The preferred particles of the invention are coded particles or beads which comprise an RF tag carried on an inert material in the form of a silicon chip, and a synthesis polymer on which the compounds of the library are synthesised.

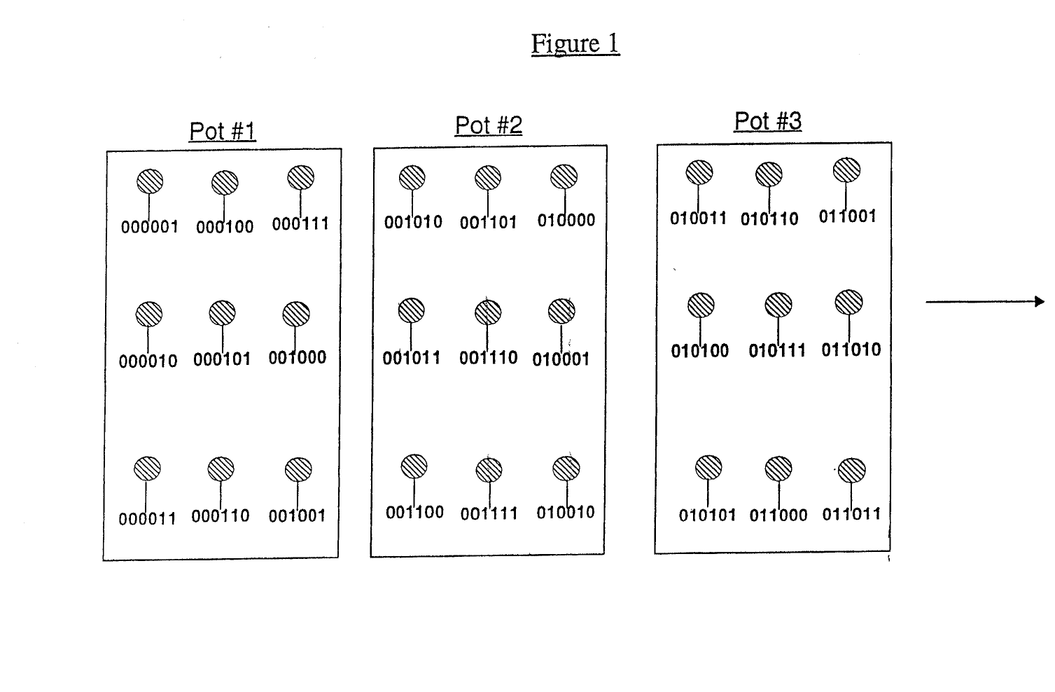

RF or radio tags are silicon integrated circuits which incorporate a memory for storing a code, an antenna for communication, and control circuitry. The memory may be read only, read and write, or a combination of the two. The tags are used in conjunction with a reader unit which transmits a high power electromagnetic field to the tags. Tags may be self-powered from an internal battery. However, of particular relevance are remotely powered tags, which obtain their power from the transmitted electromagnetic field via an inductive coil, which functions as the antenna. The field is received by the tag and converted to a power supply to run the tag circuitry. The tag can then convey its identification data to the reader by reference to the code stored in memory. The conveyance of the data from the tag to the reader may be by transmission of a signal from the tag, or more commonly, by circuits on the tag modulating the power absorbed from the field provided by the reader. In current generation, inductively coupled radio tags, the coil is usually external to the silicon chip, as shown in FIG. 1. This allows a reading range of between 10 cm and 1 m to be achieved. These tags have been developed for applications such as implantable transponders for identifying farm or laboratory animals. Here, the range is more important than achieving a very small sized tag. The size of these tags is typically 8 mm by 1 mm in area, excluding the external coil, which commonly has a ferrite rod or similar core, and they would include programmable memory and possibly even a temperature sensor. Other existing rf tags are used for mass-transit revenue collection. In these systems a very thin structure is commonly required, to allow the structure to be contained within a credit card thickness. In these systems a ferrite core generally cannot be provided, but the coil area can be considerably greater—typically occupying an area of order 60×30 mm, with as many as 700 turns of wire.

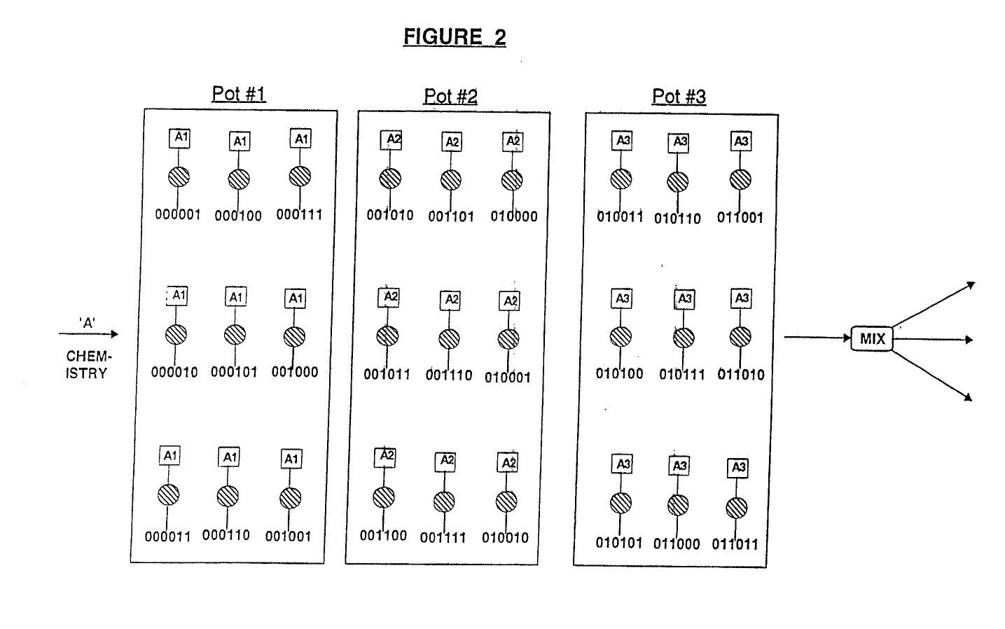

Writing to, or programming a radio tag requires considerably more powers simply reading from it, and programmable memory consumes more space on the silicon chip than read only memory. Therefore, by limiting the range and functionality of a radio tag, it is possible to decrease the size of the tag circuitry and incorporate the inductive coil onto the chip itself. Since the radiated power will fall with at least the square of the distance it is envisaged that a read only radio tag with a range of about 1 mm would have an approximate size of 1 mm by 1 mm area, and incorporate an on chip coil. This is shown schematically in FIG. 2. Such a tag may be encapsulated in an inert glass cored bead, or fabricated in a composite synthesis particle as a flat structure with associated resin.

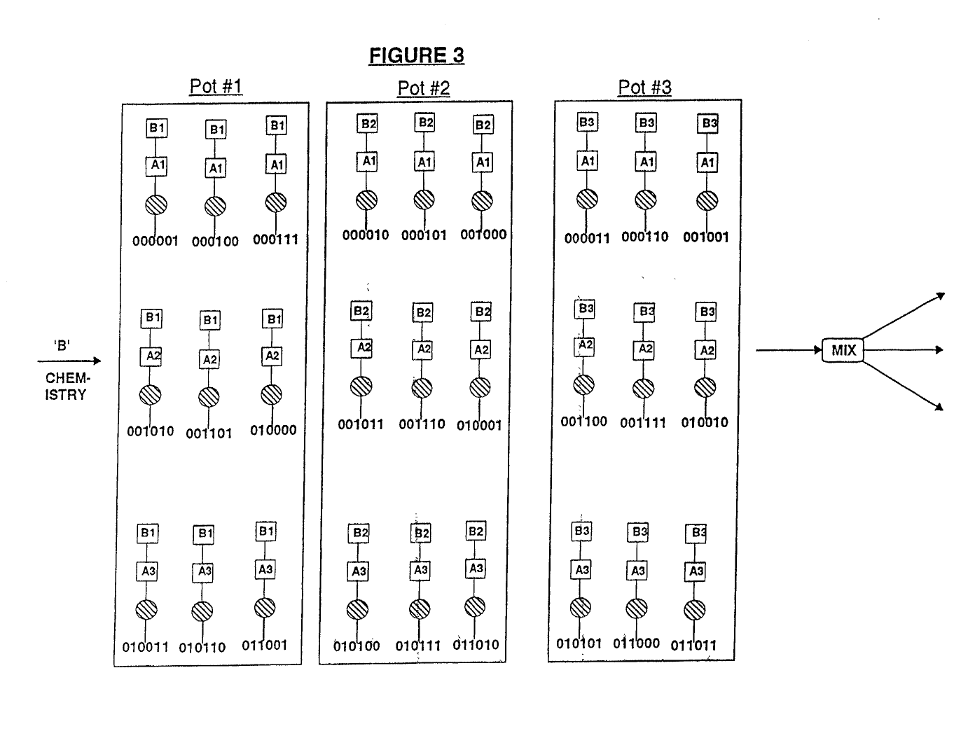

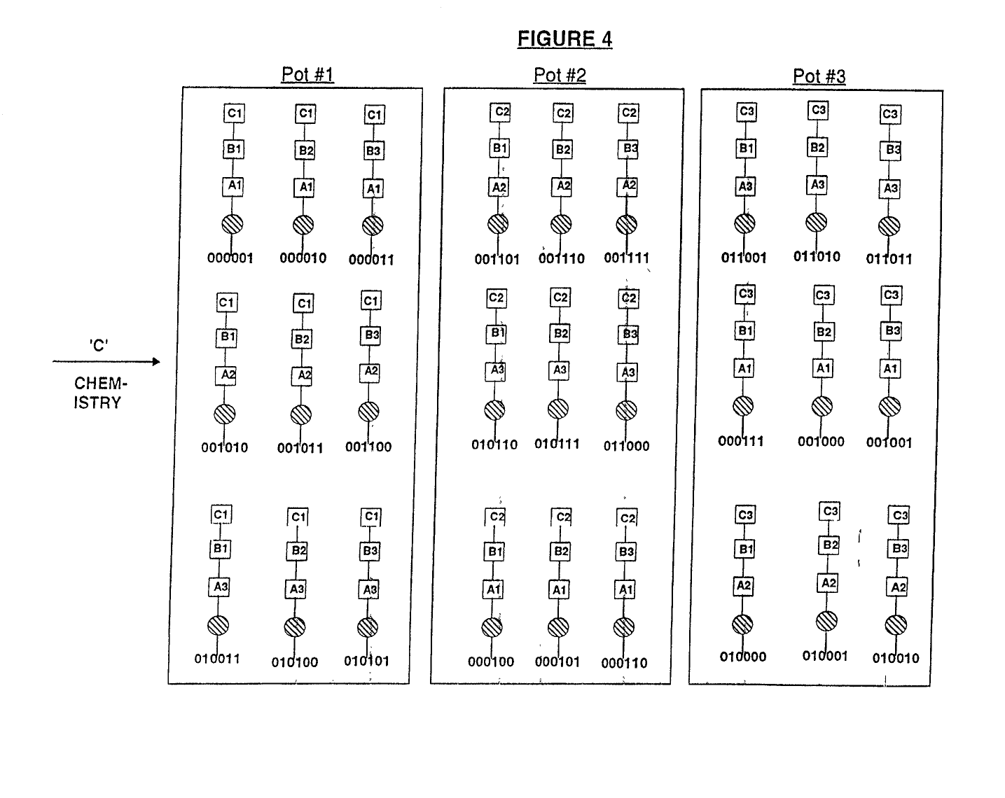

Thus, according to the current invention, RF tags applied to the individual solid phase particles utilised in combinatorial, compound library chemistry can be implemented with the silicon chip and the bead being designed as a complementary pair in a so-called ‘composite synthesis particle’ or bead. In a preferred embodiment the composite bead would comprise a flat-faced bead consisting of a thin plate or ‘substrate’, to hold the silicon integrated circuit, and a printed, or grafted coating of a suitable solid support, or ‘resin’ for compound library synthesis. A generalised composite synthesis particle is shown diagrammatically in FIG. 3. These particles may also contain attachment features that ensure that the two layers do not come apart during any ensuing processes. Such features may be for example lips, or pegs in the form of mushrooms or inverted pyramids that are built into the substrate during manufacture. Diagrammatic representations of composite particles including such ‘raised structures’ together the synthesis support to the tag are shown in FIG. 4

The silicon integrated circuit, optionally including raised structures, will be produced in the form of a wafer typically of 100-200 mm in diameter, using photolithographic techniques which are commonplace in the microelectronics industry and as such would be familiar to the artisan of ordinary skill from that area. Since silicon devices are normally fabricated on relatively thick wafers the wafer would be thinned by grinding away the back surface ro leave a wafer of perhaps 150-200 μm in thickness. The purpose for such thinning being commonly applied, in for example the fabrication of so-called ‘smart-cards’ and being familiar to those appropriately skilled. The wafer may optionally be passivated with a chemically inert layer such as a deposited glass or polyimide. Attachment of the synthesis support during particle manufacture is preferably achieved by spin coating the thinned silicon wafer with suitable polymer or by dispensing droplets of polymer by means of, for example, an ink-jet device, onto the nascent chips still held on the wafer prior to ‘dicing’ and release of the individual chips. Alternatively, the chips could be coated with synthesis polymer after release from the wafer.

Particularly preferred articles of the invention are coded particles or beads which comprise both a read-only RF tag carried on an encapsulated silicon chip, which is between 0.5 mm×0.5 mm and 2 mm×2 mm in area, and which incorporates within the built in circuitry an inductive coil, and an associated synthesis polymer on which the compounds of the library are synthesised.

The encoded radiofrequency particles of the invention allow bigger libraries to be made more conveniently. Also, the radio tag and the synthesis polymer are physically attached one to the other rather than being merely associated by being sealed together in a container. If the container is at all damaged, then the tag and the uncoded synthesis particles could become separated and the process would fail.

By “encapsulated silicon chip” we mean an integrated circuit, fabricated using silicon wafer technology as is well known in the art to include an inductive coil, that is either fully encapsulated by or partially coated with an inert protective material that is robust and inert to the chemistry of compound library production. Preferred materials include glass and polyimide. Particularly preferred is glass. The protective material can be attached to the silicon chip either by the encapsulation process, or by a covalent linkage process. A particularly preferred covalent linkage would be via siloxy bridges between the surface silicon oxide of the tag and the glass coating/encapsulation material.

The synthesis polymer or ‘resin’ portion of the beads is conveniently any species which may be used as a solid support for chemical library synthesis, for example in split-synthesis processes. The synthesis support is inherently adapted, by way of its chemical structure, for reaction with and/or preparation of chemical compounds, or is treated with suitable reagents to make it amenable to combinatorial chemistry. Solid supports consisting of polystyrene provided with chloromethyl, and/or aminomethyl, and/or hydroxymethyl groups, are particularly preferred. The resin portion may also be optionally cross-linked.

By way of non-limiting example the resin portion of an RF bead is comprised of chloromethylpolystyrene resin which has been, coated or grafted onto, or otherwise inextricably associated with the ‘substrate’ of the particle, which carries the RF code. Using the pendant chloromethyl groups a compound library is then created by a tracked split-synthesis procedure.

A preferred method is coating of the wafer with the resin material, or a precursor to the resin, by the method of spin-coating. Those skilled in the art will recognise this technique as being well-suited to the deposition of films of from <1-100 μm in thickness. The material to be coated being either the resin or a pre-cursor to the resin optionally contained with a suitable solvent or dispersion. Following spin coating any solvent would be displaced, for example by heating, and if necessary a thermal, or photo, initiated curing process would be used to complete the resin formation. Following this stage, or optionally in parallel if a photo initiated cure is employed, the resin may be patterned such that the dicing operation does not cut through the resin. Such patterning may, depending on the resin formulation required, be advantageous in preventing lifting of the resin edge. Optionally, the method of Reactive Ion Etching may be used, which together with techniques such as an erodable etch mask, can allow the resin edge to be profiled rather than cut square. It will be recognised that this same processed may be used to selectively remove resin at very fine scale, and could be used to texture the surface or otherwise provide for stress relief in the film.

The preferred RF tags of the present invention are of such a size that interrogation of the codes in ROM can be achieved remotely, in other words without direct contact, only over a short distance. To cope with this very small range, we disclose different methods of handling the beads. They may be sent one at a time through a very narrow tube, past a reader unit, or they may be handled by a robotic pick up tool incorporating the reader unit. Particularly preferred is the use of a robotic pick-up tool which incorporates a close proximity reader unit in the head.

The use of robotic pick-and-place machines is known in the microelectronics industry, most commonly in the placement of surface mount compents. Such components are typically resistors and capacitors taking the form of flat, ceramic parts—variously known as 0402, 0805, 1206 etc., with resistive components and individual diodes also being available in a cylindrical form known as MELF or micro-MELF—and active devices such as individual diodes and transistors taking the form of plastic moulded packages such as SOT-23, integrated circuits packages such as SO-8, PGA and so on. These devices are of small size, with the 0402 device being only, approximately, 1 mm×0.5 m in extent. The industry has developed robots able to manipulate these packages at high-speed with precise placement to predefined locations on, for example, aprited, circuit board. With the increasing sophistication of the devices to be manipulated, both in terms of the number of connections and the reduction in the size of individual connections and spacing between connections, the industry has adopted the use of video cameras and pattern recognition systems to ensure the accurate placement of complex components. Machines which are commonly employed in the microelectronics industry use a vacuum pick up to hold the components. The speed of placement varies with the mox of components and the size of the board onto which they are to be placed. However, the quoted throughput in commonly in the range of a few hundred to perhaps 30,000 components placed per hour.

Whilst we do not wish to be bound by theoretical considerations, we believe that a throughput of about 1 bead per second may be achieved by machines typical of those currently available.

By way of non-limiting example, we position ideally within the pick up head of a pick and place machine, at least the ‘front-end’ electronics of an rf reader device. Using this reader device the individual particles are read and the code returned is used to determine the locus to which the particle should be moved. It is apparent that this ‘on-the-fly’ determination of the particle destination is a departure from the general microelectronic use of a pick and place machine. Changes to the operating software may be required but this is rot believed to be an undue burden to the skillen artisan. Thus in a further aspect of the invention we provide a method for the preparation of an RF encoded chemical library, wherein the coded particles are ‘directed and recorded’ or ‘tracked’ to particular loci by a robotic pick-up tool. And in a further aspect of the invention we provide a method for the preparation of an RF tagged chemical library, which method comprises synthesising the library on a plurality of RF coded particles, each of which is provided with a discrete RF tag, so as to provide a chemical library comprising a plurality of solid supports to each of which is attached at least one member of the library and one RF tag.

One particularly preferred embodiment of this method, optionally using a manipulative robotic device with a reader unit in its head, is the use of a single coded particle for each target library structure required.

For example, consider a library of, say, 27,000 compounds, which is to be made by a tracked split-synthesis process using 30 primary diversity substituents, or building blocks, 30 secondary building blocks and 30 tertiary building blocks (ie. a 30×30×30 library). One would start with 27,000 unique composite synthesis particles. Into each of the 30 primary reaction vessels would be tracked 900 synthesis particles. By use of the robotic pick-up tool incorporating the reader unit the very act of picking up each individual bead would allow the reader unit to come into sufficiently close proximity to the bead that the code held in the ROM of the tag could easily be interrogated. The first stage of the library synthesis may be performed, and the particles would be recovered then deposited and tracked into secondary reaction vessels such that no more than 30 particles that were in any primary reaction vessel are placed in the same secondary reaction vessel. Again, use of the robotic tool plus close proximity reader unit makes this tracking process facile. The second stage of the library synthesis may then be performed, and again the particles would be recovered, deposited and tracked into tertiary reaction vessels. This time, however, it is an essential part of the placement process that no two particles that have been in the same primary and secondary reaction vessels are deposited in the same tertiary vessel. The third and final stage of library synthesis would then be performed, and again the particles would be recovered. Provided no two particles have passed through the same three reaction vessels, and there have been no particle losses, there will be 27,000 compounds attached to 27,000 composite synthesis particles. A simplified version of the above process is described for a library of 27 discrete compounds, 27 discrete coded synthesis particles, 3 primary ‘A’ building blocks, 3 secondary ‘B’ building blocks, and 3 tertiary ‘C’ building blocks in Scheme 1. Such a directed process thus involves the active steering of the discrete beads down RF code-specific and, for any one bead in the defined library unique, process paths resulting in a single compound por RF, ed particle. It is a process that is considerably assisted by the use of robotic pick-up tools which incorporate close proximity reader units. A further advantage to the use of a pick-and-place machine of the type described above in this process is that at the end of the synthesis it allows, if desired, for the convenient selection of one or more defined subsets of the library for special treatment or further processing. This aspect of the method provides a further key embodiment to the method of the invention.

It should be noted that synthesis of chemical libraries on the RF coded particles of the invention may comprise any convenient number of individual reaction steps.

The chemical libraries may comprise any convenient number of individual members, for example tens to hundreds to thousands to millions etc., of suitable compounds, for example peptides, peptoids and other oligomeric compounds (cyclic or linear), and template-based smaller molecules, for example benzodiazepines, hydantoins, biaryls, carbacyclic and polycyclic compounds (eg. naphthalenes, phenothiazines, acridines, steroids etc.), carbohydrate and amino acids derivatives, dihydropyridines, benzhydryls and heterocycles (eg. triazines, indoles, thiazolidines etc.). The numbers quoted and the types of compounds listed are illustrative, but not limiting. Whatever the size of the library, convenient libraries may comprise less than 1000, for example less than 500, less than 200, less than 100, less than 50 or less than 20 compounds per library vessel.

Preferred compounds are chemical compounds of low molecular weight and potential therapeutic agents. They are for example of less than about 1000 daltons, such as less than 800, 600 or 400 daltons.

Any convenient biological of interest such as a receptor, enzyme or the like may be contacted with the chemical library as above in an assay or test system apparent to the scientist of ordinary skill.

Advantages of the libraries of the this invention include:

(i) the relative ease and high fidelity with which the RF codes can be produced;

(ii) the RF coded particles are generated prior to any chemical synthesis being undertaken, so no time is taken up introducing tag information during the library synthesis process;

(iii) the coded particles may be rapidly read and checked before any synthesis is undertaken, any beads carrying unreadable codes can be rejected, thus allowing the in process bead reading to be of even higher fidelity;

(iv) close coupling between the tag and the chemistry allows tracking at all stages eg. through all process steps, library storage and screening steps.

The use of manipulative robotic devices such as pick-and-place machines, and particularly one that is modified such that a close proximity reader unit within the pick-up tool is able to read the rf tags on the devices and, using suitable control software, direct them to an appropriate locus, has a number of advantages:

(i) the ability to form a combinatorial library with one bead:one identified compound;

(ii) the ability to retain that identification through library storage and screening stages;

(iii) the ability to select subsets of the library of controlled diversity to minimise the number of compounds screened; and

(iv) the ability to subsequently select alternate subsets clustered around known compounds of interest without further synthesis steps.

In summary, by use of an RF encoded particle to assist in bead identification,and tracking, and a manipulative robotic pick-up tool, particularly a pick-up tool containing a close proximity reader unit to assist in the tracking and deposition of any particular labelled bead through each stage of a split-synthesis, one-compound-one-bead library generation process, one can conveniently associate, at the end of the process, a single chemical structure with a single RF code. The ability to utilise manipulative robotic devices to assist in the establishment of that association has considerable utility for the assaying and deconvoluting, or decoding of compound libraries.

The invention will now be illustrated but not limited by reference to the following Figures wherein:

FIGS. 1-4 show the processes involved in the generation of a model, tagged library of 27 discrete compounds on 27 discrete beads. The 27 discrete beads each carry a unique tag, in this case indicated by a 6-bit binary code, which numbers the beads from 1 to 27.

FIG. 1 shows the 27 discrete beads in pots #1, #2 and #3 prior to application of chemistry “A”.

FIG. 2 shows the application of chemistry “A” to the library and subsequent mixing of the contents of pots #1, #2, and #3. The resulting mixture is divided into the three pots.

FIG. 3 shows the application of chemistry “B”, to the library and subsequent mixing of the contents of pots #1, #2, and #3. The resulting mixture is divided into three pots.

FIG. 4 shows the application of chemistry “C”to the library and the library compounds so obtained.

The diversity elements introduced during the various (‘A’, ‘B’ and ‘C’) chemistry processes are indicated by the boxed indicators (A1, A2, B3, C3 etc.) which are attached to the hatched circles, which in turn represent the synthesis particles. The term ‘MIX’ includes both the recombining of the 27 particles and their redistribution into the 3 further pots, or reaction vessels in preparation for the next stage in the library synthesis.

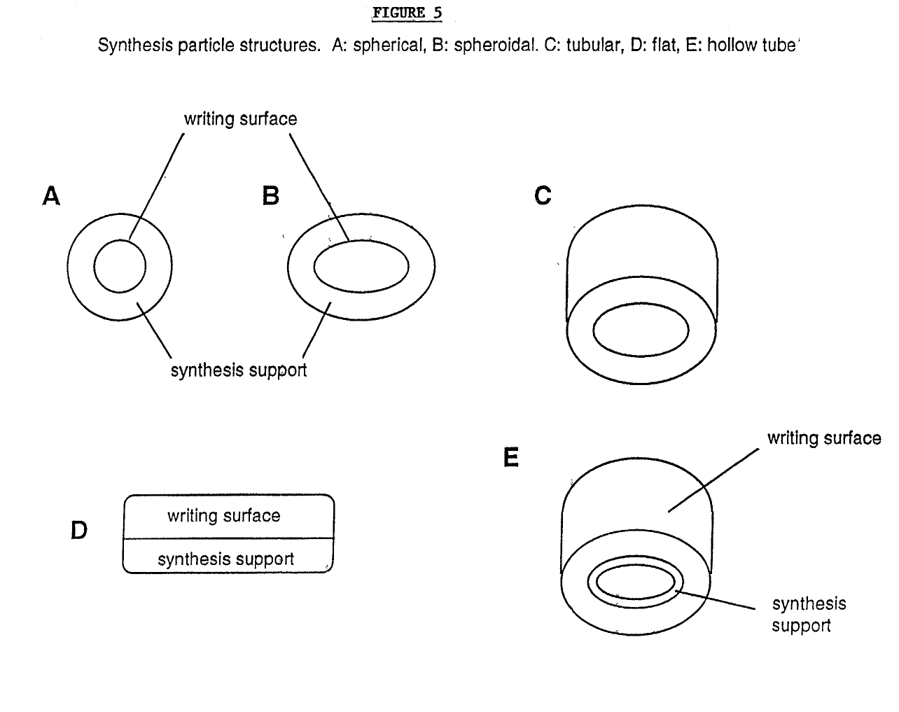

FIG. 5 shows synthesis particle structures comprising a synthesis support and a writing surface. In particular FIG. 5a shows spherical structures, FIG. 5b shows spheroid structures, FIG. 5c shows discoid structures, FIG. 5d shows flat layered structures and FIG. 5e shows hollow tube structures.

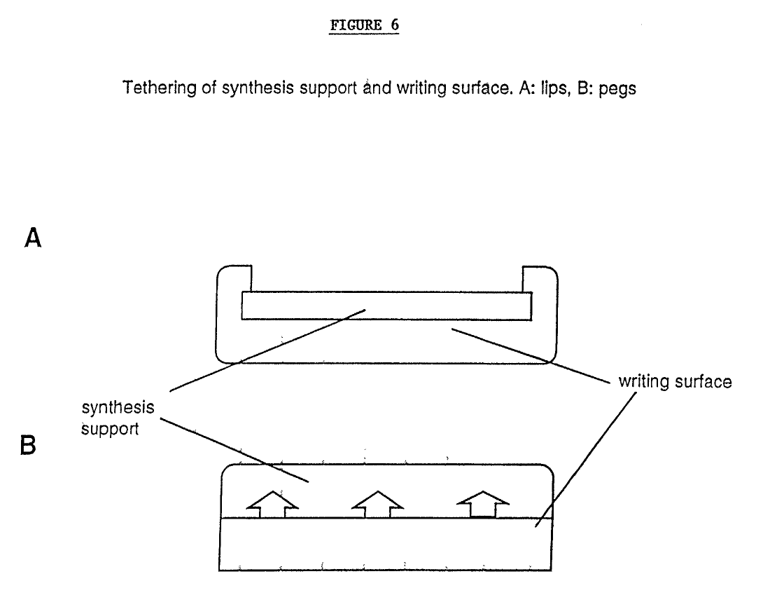

FIG. 6 shows two ways in which the synthesis support and writing surfaces of a synthesis particle way be tethered. FIG. 6a shows the use of lips and FIG. 6b shows the use of pegs.

1. A radiofrequency encoded chemical library synthesis particle which comprises a read-only radiofrequency tag linked to a solid phase.

2. A synthesis particle as claimed in claim 1 wherein the radiofrequency tag comprises EEPROM memory.

3. A synthesis particle as claimed in claim 1 wherein the radiofrequency tag comprises hard wired links to provide a read only memory.

4. A synthesis particle as claimed in any previous claim wherein the read-only radiofrequency tag is remotely-powered.

5. A synthesis particle as claimed in any previous claim wherein the read-only radiofrequency tag has an area of 2 mm² or smaller.

6. A synthesis particle as claimed in any one of the previous claims wherein the read-only radiofrequency tag is linked to the solid support so as to form an integral composite particle.

7. A synthesis particle as claimed in claim 6 wherein the tag is firstly encapsulated in an inert protective material.

8. A synthesis particle as claimed in claim 6 wherein the tag is associated with resin as a substantially flat faced structure.

9. A chemical library immobilised on a plurality of radiofrequency encoded synthesis particles according to any one of the previous claims, each particle having one or more members of the library on it and having a unique radiofrequency code.

10. A chemical library as claimed in claim 9 wherein each particle has only one member of the library on it.

11. A method for the preparation of a chemical library as claimed in claim 9 or claim 10 wherein the movement of each particle has been tracked during synthesis by reference to its individual tag.

12. A method as claimed in claim 11 wherein the synthesis particles are tracked using a robotic pick and place tool.

13. The use of a chemical library as claimed in claim 9 or claim 10 in screening methods to identify compounds which modulate the activity of a biological of interest.

14. The use as claimed in claim 13 wherein robotic apparatus is used to select library members on a random or directed basis for use in the screening methods.

15. The use as claimed in claim 14 wherein the directed basis is a structure/activity relationship.

16. A robotic “pick and place” machine adapted to read synthesis particles as claimed in any one of claims 1-8.
